(19) United States
(12) Patent Application Publication
(10) Pub. No.: US 2015/0359926 A1
(43) Pub. Date: Dec. 17, 2015

(54) Methods And Compositions For Medical Articles Produced From Proteinaceous Compounds

(71) Applicants: Curtis E. Jones, Savannah, GA (US); John P. Kennedy, Pooler, GA (US)

(72) Inventors: Curtis E. Jones, Savannah, GA (US); John P. Kennedy, Pooler, GA (US)

(21) Appl. No.: 14/307,129

(22) Filed: Jun. 17, 2014

(51) Int. Cl. A61L 26/00; C12N 9/64; A61L 27/22; A61L 24/10; A61L 24/00

(52) U.S. Cl. CPC A61L 26/0085; A61L 26/0047; A61L 26/0066; A61L 24/108; A61L 24/0036; A61L 24/0015; A61L 27/227; C12N 9/6424; A61L 2300/404; A61L 2400/04

(57) ABSTRACT

The invention disclosed herein provides compositions and methods for biocompatible biomaterials with improved control of microorganisms, improved biocompatibility, lower toxicity, and reduce vCJD transmission potential. These combined benefits cascade to provide improved efficacy, improved patient compliance and improved performance, while limiting clinical complications in treatment.

### BACKGROUND OF THE INVENTION

1. Field of the Invention

The present invention relates to the field of biomaterials and supportive devices for medical applications. More specifically, the present invention relates to compositions and production methods of proteinaceous materials, including foam dressings, foam sponges and biomaterial devices.

2. Background of the Invention

Biomaterials are commonly used in the treatment and maintenance of acute and chronic wounds of the body as well as for tissue implants, sealants and barriers. Hence, materials such as collagen and gelatin have been utilized as biocompatible materials to aid in the establishment and maintenance of a favorable environment for tissue growth and repair. On a therapeutic level, these materials generally improve fluid homeostasis and provide biocompatible matrices for tissue growth and migration. On a physical level, they serve as a secondary covering to protect and limit access to the wound from the external environment. The present invention discloses the construction and utilization of inert and bioactive peptides and proteins to produce medical articles in the form of solidified foams, pads, and granular or multiparticulate constructs for application within or upon bodily tissues. In addition to the benefits of traditional materials, the present compositions and methods, through the passive release of bioactive molecules and substances as well as the option of delivery of beneficial pharmaceutical agents, display the added benefit of altering the local environment within the wound in such a manner to be conducive to tissue growth while inhibiting opportunistic microorganisms generally detrimental medical health.

Similar devices in the prior art comprised of gelatin and collagen have disadvantages depending on their specific embodiment, including (a) lacking, or limited, control of microorganisms, (b) lower biocompatibility, (c) higher toxicity and (d) if bovine sourced, the possibility of transferring Creutzfelt-Jacob disease (vCJD).

Consequently, a need has been demonstrated for the invention which provides compositions and methods for biocompatible biomaterials with at least one of the following: (a) improved control of microorganisms, (b) improved biocompatibility, (c) lower toxicity, and (d) no vCJD potential.

### RELATED ART

A search of the prior art did not disclose any patents that read directly on the claims of the instant invention; however, the following references were considered related.

| Number | File Date | Inventor(s) |
|---|---|---|
| 4,331,547 | November 1980 | Scotts et al. |
| 4,336,258 | June 1982 | Blum |
| 4,472,840 | September 1982 | Jefferies |
| 4,394,370 | July 1983 | Jefferies |
| 4,412,947 | November 1983 | Cioca |
| 4,430,760 | February 1984 | Smestad |
| 4,614,794 | September 1986 | Easton et al. |
| 4,623,553 | November 1986 | Ries et al. |
| 4,642,117 | February 1987 | Nguyen et al. |
| 4,925,924 | October 1987 | Silver et al. |
| 4,789,401 | December 1988 | Ebinger et al. |
| 4,834,734 | May 1989 | Morganti |
| 4,865,602 | September 1989 | Smestad et al. |
| 5,138,030 | October 1989 | Pachence |
| 4,888,366 | December 1989 | Chu et al. |
| 4,890,612 | January 1990 | Kensey |
| 4,948,540 | August 1990 | Nigam |
| 5,110,604 | May 1992 | Chu et al. |
| 5,382,285 | April 1993 | Morrison |
| 5,360,828 | March 1994 | Morrison |
| 5,819,748 | August 1994 | Pfirrmann |
| RE35399 | November 1994 | Eisenberg |
| 5,624,463 | April 1997 | Stone et al. |
| 6,355,699 | June 1999 | Vyakarnam et al. |
| 5,972,385 | October 1999 | Liu et al. |
| 5,997,896 | December 1999 | Carr, Jr. et al. |
| 6,110,484 | August 2000 | Sierra |
| 6,183,498 | February 2001 | Devore et al. |
| 6,294,187 | September 2001 | Boyce et al. |
| 6,296,667 | October 2001 | Johnson et al. |
| 6,733,774 | January 2002 | Stimmeder |
| 7,098,315 | January 2002 | Schaufler et al. |
| 6,399,380 | June 2002 | Li |
| 2002/0183855 | December 2002 | Yamamoto et al. |
| 2003/0012805 | January 2003 | Chen et al. |
| 7,241,316 | July 2003 | Evans et al. |
| 7,223,386 | September 2003 | Bott et al. |
| 2004/0142037 A1 | September 2003 | Engelmayer et al. |

### SUMMARY OF THE INVENTION

The purpose of the invention is to provide compositions and methods for biocompatible biomaterials with at least one of the following advantages over the prior art: (a) improved control of microorganisms, (b) improved biocompatibility, (c) lower toxicity, and (d) no vCJD potential. These combined benefits cascade to provide improved efficacy, improved patient compliance and improved performance, while limiting clinical complications in treatment.

In one embodiment of the invention, a bioactive protein and adjunct additives are processed to exact a suspension containing gaseous inclusions or bubbles. The gaseous inclusions or bubbles may be imparted by mechanical means through vigorous agitation, homogenization and/or direct injection of gaseous products or by chemical means such as effervescent chemical or emulsification reactions. This composition is then processed in a manner to remove the liquid or fluid character and produce an article possessing a solidified structure with the rigid or semi-rigid characteristics of commonly made and used closed cell and open cell foam products. This may be achieved by the addition of energy in the form of heat or irradiation, by chemical means through the use of commonly utilized reactive cross-linking agents, and/or lyophilization. The articles may be further processed through sizing and packaged in a plurality of formats for therapeutic applications in medicine. Upon application to tissues, the system manages exudate, releases bioactive molecules beneficial to the process of healing, seals tissues, aids in the control and reduction of opportunistic bacteria, and serves as a primary cushion for wounds.

A first aspect is a proteinaceous foam composition and method of production that provides a preferred structural framework for use as foam dressings, foam sponges including hemostatic sponges, and biomaterial devices useful as tissue sealants and/or barriers. The composition and methods comprise an amino acid containing compound of natural, synthetic or recombinant origin selected from the group of proteins, glycoprotein, peptides, poly amino acids, protein hydrolysates, peptide hydrolysates, derivatives of this group and any combination thereof, and at least one augmentative polymer. Particularly useful amino acid containing compounds are albumin, gelatin and collagen. Particularly useful augmentative polymers are chitosan, glucosamine, N-acetyl glucosamine, hyaluronic acid, sulfoglucosamine, glycosylamine, and galactosamine.

In broad terms, a preferred embodiment of the composition and methods are further comprised of at least one secondary component selected from the group of an adjunct compound, an anti-infective, a crosslink augmentation agent, and a crosslinking-agent.

One advantage of the invention is that the augmentative polymer promotes the formation of the desired final physical structure, function and/or lessens toxicity, including lessening the amount of crosslinking-agent, by providing additional reactive sites than those inherent to the amino acid containing compound.

Another advantage of the invention is that the adjunct compound can promote the formation and retention of the desired final physical structure by stabilizing the liquid, preserving the composition, plasticizing the composition, and/or enhancing the viscosity.

Another advantage of the invention is that the anti-infective can limit, arrest or reduce the growth, attachment, colonization or quantity of infective micro organisms, including planktonic or biofilm phenotypes such as pathogenic and nonpathogentic bacteria, viruses, fungi, and yeasts.

Another advantage of the invention is that the crosslinking-agent can chemically react with the amino acid containing compound and secondary components to form crosslinks that provide the composition the desired final physical structure.

Another advantage of the invention is that the crosslink augmentation agent can promote the formation of the desired final physical structure, function or lessen toxicity through the potentiation of crosslinks, which lessen the total crosslinking-agent required, thereby lessening toxicity and improving biocompatibility.

Another aspect is a proteinaceous foam composition and method of production based on lactoferrin that provides a preferred structural framework for use as foam dressings, foam sponges including hemostatic sponges, and biomaterial devices useful as tissue sealants and/or barriers. The composition and methods comprise lactoferrin, or derivatives thereof, of synthetic or recombinant origin.

In broad terms, a preferred embodiment of the composition and methods are further comprised of at least one secondary component selected from the group of an augmentative polymer, an adjunct compound, an anti-infective, a crosslink augmentation agent, and a crosslinking-agent.

One advantage of the invention is that the augmentative polymer promotes the formation of the desired final physical structure, function and/or lessens toxicity, including lessening the amount of crosslinking-agent, by providing additional reactive sites than those inherent to the amino acid containing compound.

Another advantage of the invention is that the adjunct compound can promote the formation and retention of the desired final physical structure by stabilizing the liquid, preserving the composition, plasticizing the composition, and/or enhancing the viscosity.

Another advantage of the invention is that the anti-infective can limit, arrest or reduce the growth, attachment, colonization or quantity of infective micro organisms, including planktonic or biofilm phenotypes such as pathogenic and nonpathogentic bacteria, viruses, fungi, and yeasts.

Another advantage of the invention is that the crosslinking-agent can chemically react with the amino acid containing compound and secondary components to form crosslinks that provide the composition the desired final physical structure.

Another advantage of the invention is that the crosslink augmentation agent can promote the formation of the desired final physical structure, function or lessen toxicity through the potentiation of crosslinks, which lessen the total crosslinking-agent required, thereby lessening toxicity and improving biocompatibility.

In another embodiment of the invention, a bioactive protein and adjunct additives are processed to produce multiparticulates. The multiparticulates may be imparted by physical means through emulsification or homogenization followed by crosslinking to form a suspension, extrusion and spray drying, depending on the desire final structure. Upon application to tissues, the system manages exudate, releases bioactive molecules, and aids in the control and reduction of opportunistic bacteria.

A first aspect is a proteinaceous multiparticulate composition and methods of production that provide a preferred structural framework for use as multiparticulate biomaterial devices. The composition and methods comprise an amino acid containing compound of natural, synthetic or recombinant origin selected from the group of proteins, glycoprotein, peptides, poly amino acids, protein hydrolysates, peptide hydrolysates, derivatives of this group and any combination thereof, and at least one augmentative polymer. Particularly useful amino acid containing compounds are albumin, gelatin and collagen. Particularly useful augmentative polymers are chitosan, glucosamine, N-acetyl glucosamine, hyaluronic acid, sulfoglucosamine, glycosylamine, and galactosamine.

In broad terms, a preferred embodiment of the composition and methods are further comprised of at least one secondary component selected from the group of an adjunct compound, an anti-infective, a crosslink augmentation agent, and a crosslinking-agent.

One advantage of the invention is that the augmentative polymer promotes the formation of the desired final physical structure, function and/or lessens toxicity, including lessening the amount of crosslinking-agent, by providing additional reactive sites than those inherent to the amino acid containing compound.

Another advantage of the invention is that the adjunct compound can promote the formation and retention of the desired final physical structure by stabilizing the liquid, preserving the composition, plasticizing the composition, and/or enhancing the viscosity.

Another advantage of the invention is that the anti-infective can limit, arrest or reduce the growth, attachment, colonization or quantity of infective micro organisms, including planktonic or biofilm phenotypes such as pathogenic and nonpathogentic bacteria, viruses, fungi, and yeasts.

Another advantage of the invention is that the crosslinking-agent can chemically react with the amino acid containing compound and secondary components to form crosslinks that provide the composition the desired final physical structure.

Another advantage of the invention is that the crosslink augmentation agent can promote the formation of the desired final physical structure, function or lessen toxicity through the potentiation of crosslinks, which lessen the total crosslinking-agent required, thereby lessening toxicity and improving biocompatibility.

Another aspect is a proteinaceous multiparticulate composition and methods of production based on lactoferrin that provide a preferred structural framework for use as multiparticulate biomaterial devices. The composition and methods comprise lactoferrin, or derivatives thereof, of synthetic or recombinant origin.

In broad terms, a preferred embodiment of the composition and methods are further comprised of at least one secondary component selected from the group of an augmentative polymer, an adjunct compound, an anti-infective, a crosslink augmentation agent, and a crosslinking-agent.

One advantage of the invention is that the augmentative polymer promotes the formation of the desired final physical structure, function and/or lessens toxicity, including lessening the amount of crosslinking-agent, by providing additional reactive sites than those inherent to the amino acid containing compound.

Another advantage of the invention is that the adjunct compound can promote the formation and retention of the desired final physical structure by stabilizing the liquid, preserving the composition, plasticizing the composition, and/or enhancing the viscosity.

Another advantage of the invention is that the anti-infective can limit, arrest or reduce the growth, attachment, colonization or quantity of infective micro organisms, including planktonic or biofilm phenotypes such as pathogenic and nonpathogentic bacteria, viruses, fungi, and yeasts.

Another advantage of the invention is that the crosslinking-agent can chemically react with the amino acid containing compound and secondary components to form crosslinks that provide the composition the desired final physical structure.

Another advantage of the invention is that the crosslink augmentation agent can promote the formation of the desired final physical structure, function or lessen toxicity through the potentiation of crosslinks, which lessen the total crosslinking-agent required, thereby lessening toxicity and improving biocompatibility.

Further aspects will become apparent from consideration of the ensuing description of preferred embodiments of the invention. A person skilled in the art will realize that other embodiments of the invention are possible and that the details of the invention can be modified in a number of respects, all without departing from the inventive concept. Thus, the following drawings and description are to be regarded as illustrative in nature and not restrictive.

### BRIEF DESCRIPTION OF THE DRAWINGS

There are no drawings.

### DESCRIPTION OF SPECIFIC EMBODIMENTS OF THE INVENTION

DEFINITIONS: As used in this description and the accompanying claims, the following terms shall have the meanings indicated, unless the context otherwise requires:

“Proteinaceous” as broadly defined and used herein, means an amino acid containing compound or composition selected from the group of proteins, peptides, poly amino acids, protein hydrolysates, peptide hydrolysates, derivatives of this group or any combination thereof.

“Crosslinking-agent” as broadly defined and used herein, means any reagent that produces a chemical reaction that forms crosslinks of proteinaceous compounds.

“Augmentative polymer” as broadly defined and used herein, means any polymer when part of a proteinaceous composition as disclosed herein, that potentiates the formation of the desired final physical structure, function or toxicity, including lessening the amount of crosslinking-agent required or residual crosslinking-agent.

“Adjunctive compound” as broadly defined and used herein, means any compound when part of a proteinaceous composition as disclosed herein, that potentiates the formation and retention of the desired final physical structure, including lessening the amount of crosslinking-agent required or residual crosslinking-agent, as a stabilizer, preservative, plasticizer or viscosity enhancer.

“Crosslink augmentation agent” as broadly defined and used herein, means any compound when part of a proteinaceous composition as disclosed herein, that potentiates the formation of the desired final physical structure, function or toxicity through the potentiation of crosslinks, including lessening the amount of crosslinking-agent required or residual crosslinking-agent.

“Foam” as broadly defined and used herein, means a material formed by trapping gas bubbles within for form cells. Foam further includes two types of distinct structure, open and closed cell types. By example, open cell foams contain primarily open pores that are interconnected and most commonly formed by the rupture of the cells during process. Open cell foams are therefore porous. By example, closed cell foams do not have interconnected pores, as the cells formed during processing are largely intact and unruptured.

“Anti-infective” when used as an adjective or adverb herein, means broadly having or exhibiting the ability to limit, arrest or reduce the growth, attachment, colonization or quantity of infective micro organisms, including planktonic or biofilm phenotypes such as pathogenic and nonpathogentic bacteria, viruses, fungi, and yeasts. When used as a noun herein, or as a noun derivative, the noun means any substance or composition having or exhibiting the ability to limit, arrest or reduce the growth, attachment, colonization or quantity of infective micro organisms, including planktonic or biofilm phenotypes such as pathogenic and nonpathogentic bacteria, viruses, fungi, and yeasts.

“Primary Dressing” when used herein shall mean any foreign material, any collection of foreign materials, or any composition of foreign materials positioned in direct contact with a wound bed. Examples include a primary dressing separating the tissue bed from a secondary dressing.

“Secondary Dressing” when used herein shall mean any foreign material, collection of foreign materials or any composition of foreign materials positioned on top of a primary dressing. Examples include wraps, tapes or dressings used to hold a primary dressing in place.

### I. Foams: Proteinaceous and Polymer Composite Derived

Broadly a proteinaceous foam composition is disclosed which provides preferred structural framework for use as foam dressings, foam sponges including hemostatic sponges, and biomaterial devices useful as tissue sealants and/or barriers.

The composition comprises an amino acid containing compound of natural, synthetic or recombinant origin selected from the group of proteins, glycoprotein, peptides, poly amino acids, protein hydrolysates, peptide hydrolysates, derivatives of this group and any combination thereof, and at least one augmentative polymer. Particularly useful amino acid containing compounds are albumin, gelatin and collagen. Particularly useful concentrations range from 2.5 to 25%. Particularly useful augmentative polymers are chitosan, glucosamine, N-acetyl glucosamine, hyaluronic acid, sulfoglucosamine, glycosylamine, and galactosamine. Particularly useful concentrations range from 0.001 to 20%.

A second best mode of the invention further comprises at least one adjunct compound to promote the formation and retention of the desired final physical structure, including lessening the amount of crosslinking-agent required or remaining (less toxicity). Useful adjunct compounds are stabilizers, preservatives, plasticizers, viscosity enhancers, and any combination thereof. Particularly useful adjunct compounds are surfactants, fatty acids, hydrogen peroxide, and poly(ethylene glycol).

A third best mode of the invention further comprises at least one anti-infective to reduce the growth, attachment, colonization or quantity of infective micro organisms, including planktonic or biofilm phenotypes of pathogenic and nonpathogentic bacteria, viruses, fungi, and yeasts. Particularly useful anti-infectives are urea, fatty acids, silver compounds, lysozyme, sugar alcohols, methylene blue, gentian violet, glycopeptides, and lipoglycopeptides.

A fourth best mode of the invention further comprises at least one crosslinking-agent to produce a chemical reaction which chemically crosslinks compounds in the composition that contain reactive sites together to form the solidified structure. Particularly useful crosslinking-agents are formaldehyde, glutaraldehyde, acetaldehyde, malonaldehyde, succinaldehyde, adipaldehyde, and dialdehyde starch. Particularly useful concentrations range from 0.001 to 10% (unreacted).

A fifth best mode of the invention further comprises at least one crosslink augmentation agent to promote the formation of the desired final physical structure, function or toxicity through the potentiation of crosslinks, including lessening the amount of crosslinking-agent required or remaining (toxicity). Particularly useful crosslink augmentation agents are polyamine compounds, resorcinol, vanillin, urea, nicotinamide, carbodiimide, and cyanamide.

One method of the invention may be operated by combining a gas with an amino acid containing compound, an augmentative polymer and at least one secondary component selected from the group of: (i) an adjunct compound, (ii) an anti-infective, (iii) a crosslink augmentation agent, and (iv) a crosslinking-agent.

The embodiments are further described by the following aspects:
1. A foam composition useful as a tissue sealant, tissue dressing, hemostatic sponge or tissue barrier comprising: (a) an amino acid containing compound of natural, synthetic or recombinant origin selected from the group of proteins, glycoprotein, peptides, poly amino acids, protein hydrolysates, peptide hydrolysates, derivatives of this group and any combination thereof and (b) an augmentative polymer.
2. A composition according to Item 1 where the amino acid containing compound is selected from the group of lysozyme, albumin, lactalbumin, bovine serum albumin, human serum albumin, gelatin, casein, collagen, fibrinogen, gliadin, an enzyme, a hydrolysates, derivatives of this group and any combination thereof.
3. The composition of Item 1 further comprising a secondary component selected from the group of: (a) an adjunct compound, (b) an anti-infective, (c) a crosslink augmentation agent, (d) a crosslinking-agent, and any combination thereof.
4. The composition of Item 3 wherein the augmentative polymer, monomer or compound contains reactive sites selected from the group of a nitrogen containing site, a sulfur containing site, or any combination thereof.
5. The composition of Item 4 wherein the augmentative polymer, monomer or compound is selected from the group of chitin, chitosan, glucosamine, N-acetyl glucosamine, hyaluronic acid, sulfoglucosamine, chondroitin, adenosine, an aminoglycoside, glycosylamine, galactosamine, a derivative of this group, and any combination thereof.
6. The composition of Item 3 wherein the adjunctive compound is selected from the group of surfactants, antioxidants, fatty acids, polyvinylpyrrolidone, polyvinyl alcohol, hydrogen peroxide, methacrylic acid polymers, and poly(ethylene glycol), carrageenen, alginates, derivatives of this group or any combination thereof.
7. The composition of Item 3 wherein the anti-infective is selected from the group of urea, a lipid compound or compounds, fatty acids, a silver compound, lysozyme, sulfonamide, sulfamethoxazole, a sugar, a sugar alcohol, xylitol, methylene blue, gentian violet, an aminoglycoside, tetracyclines, macrolides, glycopeptides, lipoglycopeptides, beta lactams, cefalosporins, quinolones, a derivative of this group, or any combination thereof.
8. The composition of Item 3 wherein the crosslinking-agent is selected from the group consisting of an aldehyde compound, a polyaldehyde compound, formaldehyde, glutaraldehyde, acetaldehyde, malonaldehyde, succinaldehyde, adipaldehyde, dialdehyde starch; glyoxal, glyoxylic acid, adipyldichloride, acrolein, N,N′-methylenebisacrylamide, diphenylphosphoryl azide, N,N′-ethylenebisacrylamide, diphenylphosphoryl azide, (poly)ethylene glycol di(meth)acrylate and functionalized (poly)ethylene glycol derivatives, ethylene glycol diglycidyl ether, glycidylmethacrylate, polyamidoamineepichlorohydrin, trimethylolpropanetriacrylate, piperazinediacrylamide, epichlorohydrin, 1,2-diol compounds, functionalized peptides and proteins, tannins, derivatives of this group or any combination thereof.
9. The composition of Item 3 wherein the crosslink augmentation agent is selected from the group of polyamine compounds, polyhydroxybenzene, resorcinol, vanillin, nicotinamide, adenosine, a derivative of this group or any combination thereof.
10. A method of producing a foam useful as a tissue sealant, tissue dressing or tissue barrier comprising: combining (a) an amino acid containing compound of natural, synthetic or recombinant origin selected from the group of proteins, glycoprotein, peptides, poly amino acids, protein hydrolysates, peptide hydrolysates, derivatives of this group and any combination thereof, (b) an augmentative polymer and (c) a secondary component selected from the group of: (i) an adjunct compound, (ii) an anti-infective, (iii) a crosslink augmentation agent, (iv) a crosslinking-agent, and any combination thereof.

### II. Foams: Lactoferrin Derived

Broadly a lactoferrin based foam composition is disclosed which provides preferred structural framework and anti-infective properties for use as foam dressings, foam sponges including hemostatic sponges, and biomaterial devices useful as tissue sealants and/or barriers.

The composition comprises lactoferrin, or derivatives thereof including lactoferricin, from natural, synthetic or recombinant origin in a solidified cellular foam structure. Particularly useful concentrations range from 2.5 to 25%.

A second best mode of the invention further comprises at least one augmentative polymer. Particularly useful augmentative polymers are chitosan, glucosamine, N-acetyl glucosamine, hyaluronic acid, sulfoglucosamine, glycosylamine, and galactosamine. Particularly useful concentrations range from 0.001 to 20%.

A third best mode of the invention further comprises at least one adjunct compound to promote the formation and retention of the desired final physical structure, including lessening the amount of crosslinking-agent required or remaining (less toxicity). Useful adjunct compounds are stabilizers, preservatives, plasticizers, viscosity enhancers, and any combination thereof. Particularly useful adjunct compounds are surfactants, fatty acids, hydrogen peroxide, and poly(ethylene glycol).

A fourth best mode of the invention further comprises at least one anti-infective to reduce the growth, attachment, colonization or quantity of infective micro organisms, including planktonic or biofilm phenotypes of pathogenic and nonpathogentic bacteria, viruses, fungi, and yeasts. Particularly useful anti-infectives are urea, fatty acids, silver compounds, lysozyme, sugar alcohols, methylene blue, gentian violet, glycopeptides, and lipoglycopeptides.

A fifth best mode of the invention further comprises at least one crosslinking-agent to produce a chemical reaction which chemically crosslinks compounds in the composition that contain reactive sites together to form the solidified cellular foam structure. Particularly useful crosslinking-agents are formaldehyde, glutaraldehyde, acetaldehyde, malonaldehyde, succinaldehyde, adipaldehyde, and dialdehyde starch. Particularly useful concentrations range from 0.001 to 10%. As known throughout the art, modification of any active protein often results in a severe diminishment, or total loss of activity. In fact, lactoferrin produced recombinantly, still maintains >90% similarity and yet is known to lose antimicrobial activity even from such a relatively small change. A priori, it was expected that crosslinking would likewise terminate the antimicrobial activity of lactoferrin, as the crosslinking process effectively denatures it, which is also known to terminate antimicrobial efficacy. However, upon testing the resulting crosslinked materials, a very significant antimicrobial effect was unexpectedly discovered, even surpassing the native lactoferrin activity for some microbes.

A sixth best mode of the invention further comprises at least one crosslink augmentation agent to promote the formation of the desired final physical structure, function or toxicity through the potentiation of crosslinks, including lessening the amount of crosslinking-agent required or remaining (toxicity). Particularly useful crosslink augmentation agents are polyamine compounds, resorcinol, vanillin, urea, nicotinamide, carbodiimide, and cyanamide.

The final product may include crosslinks with lactoferrin alone, or crosslinks with lactoferrin and any other component of the composition with reactive sites for the crosslinking agent. In fact, this is desirable when the composition includes augmentative polymers and crosslink augmentation agents to impact the physical properties of the solidified product in use.

One method of the invention may be operated by combining a gas with lactoferrin, and at least one secondary component selected from the group of: (i) an augmentative polymer, (ii) an adjunct compound, (iii) an anti-infective, (iv) a crosslink augmentation agent, and (v) a crosslinking-agent.

The embodiments are further described by the following aspects:
11. A foam composition useful as a tissue sealant, tissue dressing, hemostatic sponge or tissue barrier comprising: lactoferrin, derivatives thereof, and any combination thereof solidified into a final product.
12. The composition of Item 11 further comprising a secondary component selected from the group of: (a) an augmentative polymer, monomer, or compound with reactive groups, (b) an adjunct compound, (c) an anti-infective, (d) a crosslink augmentation agent, (e) a crosslinking-agent, and any combination thereof.
13. The composition of Item 12 wherein the augmentative polymer, monomer or compound contains reactive sites selected from the group of a nitrogen containing site, a sulfur containing site, or any combination thereof.
14. The composition of Item 13 wherein the augmentative polymer, monomer or compound is selected from the group of chitin, chitosan, glucosamine, N-acetyl glucosamine, hyaluronic acid, sulfoglucosamine, chondroitin, adenosine, an aminoglycoside, glycosylamine, galactosamine, a derivative of this group, and any combination thereof.
15. The composition of Item 12 wherein the adjunctive compound is selected from the group of surfactants, antioxidants, fatty acids, polyvinylpyrrolidone, polyvinyl alcohol, hydrogen peroxide, methacrylic acid polymers, and poly(ethylene glycol), carrageenen, alginates, derivatives of this group or any combination thereof.
16. The composition of Item 12 wherein the anti-infective is selected from the group of urea, a lipid compound or compounds, fatty acids, a silver compound, lactoferrin, lysozyme, sulfonamide, sulfamethoxazole, a sugar, a sugar alcohol, xylitol, methylene blue, gentian violet, iodine, iodophors, an aminoglycoside, tetracyclines, macrolides, glycopeptides, lipoglycopeptides, beta lactams, cefalosporins, quinolones, a derivative of this group, or any combination thereof.
17. The composition of Item 12 wherein the crosslinking-agent is selected from the group consisting of an aldehyde compound, a polyaldehyde compound, formaldehyde, glutaraldehyde, acetaldehyde, malonaldehyde, succinaldehyde, adipaldehyde, dialdehyde starch; glyoxal, glyoxylic acid, adipyldichloride, acrolein, N,N′-methylenebisacrylamide, diphenylphosphoryl azide, N,N′-ethylenebisacrylamide, diphenylphosphoryl azide, (poly)ethylene glycol di(meth)acrylate and functionalized (poly)ethylene glycol derivatives, ethylene glycol diglycidyl ether, glycidylmethacrylate, polyamidoamineepichlorohydrin, trimethylolpropanetriacrylate, piperazinediacrylamide, epichlorohydrin, 1,2-diol compounds, functionalized peptides and proteins, tannins, derivatives of this group or any combination thereof.
18. The composition of Item 12 wherein the crosslink augmentation agent is selected from the group of polyamine compounds, polyhydroxybenzene, resorcinol, vanillin, nicotinamide, adenosine, carbodiimide, cyanamide, a derivative of this group or any combination thereof.
19. A method of producing a foam useful as a tissue sealant, tissue dressing or tissue barrier comprising: combining (a) lactoferrin, derivatives thereof, and any combination thereof and (b) a secondary component selected from the group of: (i) an augmentative polymer, monomer, or compound with reactive groups, (ii) an adjunct compound, (iii) an anti-infective, (iv) a crosslink augmentation agent, (v) a crosslinking-agent, and any combination thereof.

### III. Multiparticulates: Proteinaceous and Polymer Composite Derived

Broadly a multiparticulate composition is disclosed which provides a preferred structural framework for useful as biomaterial devices, tissue implants and tissue dressings including wound dressings.

The composition comprises an amino acid containing compound of natural, synthetic or recombinant origin selected from the group of proteins, glycoprotein, peptides, poly amino acids, protein hydrolysates, peptide hydrolysates, derivatives of this group and any combination thereof, and at least one augmentative polymer in a multiparticulate structure. Particularly useful amino acid containing compounds are albumin, gelatin and collagen. Particularly useful augmentative polymers are chitosan, glucosamine, N-acetyl glucosamine, hyaluronic acid, sulfoglucosamine, glycosylamine, and galactosamine.

A second best mode of the invention further comprises at least one adjunct compound to promote the formation and retention of the desired final physical structure, including lessening the amount of crosslinking-agent required or remaining (less toxicity). Useful adjunct compounds are stabilizers, preservatives, plasticizers, viscosity enhancers, and any combination thereof. Particularly useful adjunct compounds are surfactants, fatty acids, hydrogen peroxide, and poly(ethylene glycol).

A third best mode of the invention further comprises at least one anti-infective to reduce the growth, attachment, colonization or quantity of infective micro organisms, including planktonic or biofilm phenotypes of pathogenic and nonpathogentic bacteria, viruses, fungi, and yeasts. Particularly useful anti-infectives are urea, fatty acids, silver compounds, lysozyme, sugar alcohols, methylene blue, gentian violet, glycopeptides, and lipoglycopeptides.

A fourth best mode of the invention further comprises at least one crosslinking-agent to produce a chemical reaction which chemically crosslinks compounds in the composition that contain reactive sites together into a solidified structure. Particularly useful crosslinking-agents are formaldehyde, glutaraldehyde, acetaldehyde, malonaldehyde, succinaldehyde, adipaldehyde, and dialdehyde starch.

A fifth best mode of the invention further comprises at least one crosslink augmentation agent to promote the formation of the desired final physical structure, function or toxicity through the potentiation of crosslinks, including lessening the amount of crosslinking-agent required or remaining (toxicity). Particularly useful crosslink augmentation agents are polyamine compounds, resorcinol, vanillin, urea, nicotinamide, carbodiimide, and cyanamide.

One method of the invention may be operated by combining an amino acid containing compound, an augmentative polymer and at least one secondary component selected from the group of: (i) an adjunct compound, (ii) an anti-infective, (iii) a crosslink augmentation agent, and (iv) a crosslinking-agent.

The embodiments are further described by the following aspects:
20. A multiparticulate composition useful as a biomaterial device, tissue implant, hemostatic sponge and wound dressing comprising: (a) an amino acid containing compound of natural, synthetic or recombinant origin selected from the group of proteins, glycoprotein, peptides, poly amino acids, protein hydrolysates, peptide hydrolysates, derivatives of this group and any combination thereof and (b) an augmentative polymer.
21. A composition according to Item 20 where the amino acid containing compound is selected from the group of lysozyme, albumin, lactalbumin, bovine serum albumin, human serum albumin, gelatin, casein, collagen, fibrinogen, gliadin, an enzyme, a hydrolysates, derivatives of this group and any combination thereof.
22. The composition of Item 20 further comprising a secondary component selected from the group of: (a) an adjunct compound, (b) an anti-infective, (c) a crosslink augmentation agent, (d) a crosslinking-agent, and any combination thereof.
23. The composition of Item 22 wherein the augmentative polymer, monomer or compound contains reactive sites selected from the group of a nitrogen containing site, a sulfur containing site, or any combination thereof.
24. The composition of Item 23 wherein the augmentative polymer, monomer or compound is selected from the group of chitin, chitosan, glucosamine, N-acetyl glucosamine, hyaluronic acid, sulfoglucosamine, chondroitin, adenosine, an aminoglycoside, glycosylamine, galactosamine, a derivative of this group, and any combination thereof.
25. The composition of Item 22 wherein the adjunctive compound is selected from the group of surfactants, antioxidants, fatty acids, polyvinylpyrrolidone, polyvinyl alcohol, hydrogen peroxide, methacrylic acid polymers, and poly(ethylene glycol), carrageenen, alginates, derivatives of this group or any combination thereof.
26. The composition of Item 22 wherein the anti-infective is selected from the group of urea, a lipid compound or compounds, fatty acids, a silver compound, lysozyme, sulfonamide, sulfamethoxazole, a sugar, a sugar alcohol, xylitol, methylene blue, gentian violet, an aminoglycoside, tetracyclines, macrolides, glycopeptides, lipoglycopeptides, beta lactams, cefalosporins, quinolones, a derivative of this group, or any combination thereof.
27. The composition of Item 22 wherein the crosslinking-agent is selected from the group consisting of an aldehyde compound, a polyaldehyde compound, formaldehyde, glutaraldehyde, acetaldehyde, malonaldehyde, succinaldehyde, adipaldehyde, dialdehyde starch; glyoxal, glyoxylic acid, adipyldichloride, acrolein, N,N′-methylenebisacrylamide, diphenylphosphoryl azide, N,N′-ethylenebisacrylamide, diphenylphosphoryl azide, (poly)ethylene glycol di(meth)acrylate and functionalized (poly)ethylene glycol derivatives, ethylene glycol diglycidyl ether, glycidylmethacrylate, polyamidoamineepichlorohydrin, trimethylolpropanetriacrylate, piperazinediacrylamide, epichlorohydrin, 1,2-diol compounds, functionalized peptides and proteins, tannins, derivatives of this group or any combination thereof.
28. The composition of Item 22 wherein the crosslink augmentation agent is selected from the group of polyamine compounds, polyhydroxybenzene, resorcinol, vanillin, nicotinamide, adenosine, carbodiimide, cyanamide, a derivative of this group or any combination thereof.
29. A method of producing a multiparticulate useful as a biomaterial device, tissue implant and wound dressing comprising: combining (a) an amino acid containing compound of natural, synthetic or recombinant origin selected from the group of proteins, glycoprotein, peptides, poly amino acids, protein hydrolysates, peptide hydrolysates, derivatives of this group and any combination thereof, (b) an augmentative polymer and (c) a secondary component selected from the group of: (i) an adjunct compound, (ii) an anti-infective, (iii) a crosslink augmentation agent, (iv) a crosslinking-agent, and any combination thereof.

### IV. Multiparticulates: Lactoferrin Derived

Broadly a multiparticulate composition is disclosed which provides a preferred structural framework for useful as biomaterial devices, tissue implants and tissue dressings including wound dressings.

The composition comprises lactoferrin, or derivatives thereof including lactoferricin, from natural, synthetic or recombinant origin in a solidified multiparticulate structure.

A second best mode of the invention further comprises at least one augmentative polymer. Particularly useful augmentative polymers are chitosan, glucosamine, N-acetyl glucosamine, hyaluronic acid, sulfoglucosamine, glycosylamine, and galactosamine.

A third best mode of the invention further comprises at least one adjunct compound to promote the formation and retention of the desired final physical structure, including lessening the amount of crosslinking-agent required or remaining (less toxicity). Useful adjunct compounds are stabilizers, preservatives, plasticizers, viscosity enhancers, and any combination thereof. Particularly useful adjunct compounds are surfactants, fatty acids, hydrogen peroxide, and poly(ethylene glycol).

A fourth best mode of the invention further comprises at least one anti-infective to reduce the growth, attachment, colonization or quantity of infective micro organisms, including planktonic or biofilm phenotypes of pathogenic and nonpathogentic bacteria, viruses, fungi, and yeasts. Particularly useful anti-infectives are urea, fatty acids, silver compounds, lysozyme, sugar alcohols, methylene blue, gentian violet, glycopeptides, and lipoglycopeptides.

A fifth best mode of the invention further comprises at least one crosslinking-agent to produce a chemical reaction which chemically crosslinks compounds within the composition that contain reactive sites together into a solidified structure. Particularly useful crosslinking-agents are formaldehyde, glutaraldehyde, acetaldehyde, malonaldehyde, succinaldehyde, adipaldehyde, and dialdehyde starch.

A sixth best mode of the invention further comprises at least one crosslink augmentation agent to promote the formation of the desired final physical structure, function or toxicity through the potentiation of crosslinks, including lessening the amount of crosslinking-agent required or remaining (toxicity). Particularly useful crosslink augmentation agents are polyamine compounds, resorcinol, vanillin, urea, nicotinamide, carbodiimide, and cyanamide.

One method of the invention may be operated by combining lactoferrin, and at least one secondary component selected from the group of: (i) an augmentative polymer, (ii) an adjunct compound, (iii) an anti-infective, (iv) a crosslink augmentation agent, and (v) a crosslinking-agent.

The embodiments are further described by the following aspects:
30. A multiparticulate composition useful as a biomaterial device, tissue implant, hemostatic granules and wound dressing comprising: lactoferrin, derivatives thereof, and any combination thereof.
31. The composition of Item 30 further comprising a secondary component selected from the group of: (a) an augmentative polymer, monomer, or compound with reactive groups, (b) an adjunct compound, (c) an anti-infective, (d) a crosslink augmentation agent, (e) a crosslinking-agent, and any combination thereof.
32. The composition of Item 31 wherein the augmentative polymer, monomer or compound contains reactive sites selected from the group of a nitrogen containing site, a sulfur containing site, or any combination thereof.
33. The composition of Item 32 wherein the augmentative polymer, monomer or compound is selected from the group of chitin, chitosan, glucosamine, N-acetyl glucosamine, hyaluronic acid, sulfoglucosamine, chondroitin, adenosine, an aminoglycoside, glycosylamine, galactosamine, a derivative of this group, and any combination thereof.
34. The composition of Item 31 wherein the adjunctive compound is selected from the group of surfactants, antioxidants, fatty acids, polyvinylpyrrolidone, polyvinyl alcohol, hydrogen peroxide, methacrylic acid polymers, and poly(ethylene glycol), carrageenen, alginates, derivatives of this group or any combination thereof.
35. The composition of Item 31 wherein the anti-infective is selected from the group of urea, a lipid compound or compounds, fatty acids, a silver compound, lactoferrin, lysozyme, sulfonamide, sulfamethoxazole, a sugar, a sugar alcohol, xylitol, methylene blue, gentian violet, an aminoglycoside, tetracyclines, macrolides, glycopeptides, lipoglycopeptides, beta lactams, cefalosporins, quinolones, a derivative of this group, or any combination thereof.
36. The composition of Item 31 wherein the crosslinking-agent is selected from the group consisting of an aldehyde compound, a polyaldehyde compound, formaldehyde, glutaraldehyde, acetaldehyde, malonaldehyde, succinaldehyde, adipaldehyde, dialdehyde starch; glyoxal, glyoxylic acid, adipyldichloride, acrolein, N,N′-methylenebisacrylamide, diphenylphosphoryl azide, N,N′-ethylenebisacrylamide, diphenylphosphoryl azide, (poly)ethylene glycol di(meth)acrylate and functionalized (poly)ethylene glycol derivatives, ethylene glycol diglycidyl ether, glycidylmethacrylate, polyamidoamineepichlorohydrin, trimethylolpropanetriacrylate, piperazinediacrylamide, epichlorohydrin, 1,2-diol compounds, functionalized peptides and proteins, tannins, derivatives of this group or any combination thereof.
37. The composition of Item 31 wherein the crosslink augmentation agent is selected from the group of polyamine compounds, polyhydroxybenzene, resorcinol, vanillin, nicotinamide, adenosine, a derivative of this group or any combination thereof.
38. A method of producing a multiparticulate useful as a biomaterial device, tissue implant and wound dressing comprising: combining (a) lactoferrin, derivatives thereof, and any combination thereof and (b) a secondary component selected from the group of: (i) an augmentative polymer, monomer, or compound with reactive groups, (ii) an adjunct compound, (iii) an anti-infective, (iv) a crosslink augmentation agent, (v) a crosslinking-agent, and any combination thereof.

### V. Further Methods

Embodiments disclosed above are further described by the following aspects:
39. A method of foam or multiparticulate production utilizing the composition of Item 1-38 comprising chemically reacting the composition with at least one of the secondary components to facilitate the formation and maintenance of the final structure.
40. A method of foam or multiparticulate production utilizing the composition of Item 1-38 comprising emulsifying the composition with at least one of the secondary components to facilitate the formation and maintenance of the final structure.
41. The method of Item 40 where the chemical reaction step comprises at least partially crosslinking components of the composition to facilitate the formation and maintenance of the final structure.
42. The method of Item 40 where the emulsifying step comprises at a stabilizing agent including surfactants.
43. A method of foam production utilizing the composition of Item 1-19 comprising induction of gas bubbles via mechanical force, including agitation, shaking, mixing, homogenization, and any combination thereof to impart internal structure to the composition.
44. The method of Item 43 further comprising instilling a gas, including via injection, with the mechanical force to potentiate the formation of cells.
45. The method of Item 43 wherein the gas is selected from the group consisting of nitrogen, oxygen, carbon dioxide, nitric oxide and any combination thereof.
46. A method of foam or multiparticulate production utilizing the composition of Item 1-38 further comprising lyophilizing the composition to facilitate the formation or maintenance of a more rigid the structure.
47. A method of foam or multiparticulate production utilizing the composition of Item 1-38 further comprising heating the composition to facilitate the formation or maintenance of a more rigid the structure.
48. A method of foam or multiparticulate production utilizing the composition of Item 1-38 further comprising irradiating the composition to facilitate the formation or maintenance of a more rigid the structure.
49. The method of Item 48 whereby the radiation source is provided by radio frequency, microwave, or ionizing radiation source.
50. A method of foam production utilizing the composition of Item 1-19 further comprising casting the composition into an intermediate shape via a mold.
51. The method of Item 50 whereby the mold imparts specific conformational shape.
52. The method of Item 50 whereby the mold imparts singular or a plurality of holes, protrusions and any combination thereof.

### VI. Ancillary Compositions & Methodologies

Embodiments disclosed above are further described by the following aspects:
53. The foam composition of Item 1-19 further comprising swellable compounds, particles or multiparticulates to facilitate the formation of pores within the structure of the article or system.
54. The foam composition of Item 1-19 further comprising water absorbing compounds, particles or multiparticulates to facilitate the absorptive capacity of the article or system.
55. The foam composition of Item 1-19, where the finished product has an open-cell or closed cell structure.
56. The foam composition of Item 1-19 further comprising soluble compounds, particles or multiparticulates to facilitate the formation of pores within the structure of the article or system.
57. A method of treating damaged or diseased tissues comprising application of the composition of Item 1-38 within or upon the body of a human or animal.
58. The method of Item 57 wherein the damaged tissue is a wound.
59. The method of Item 57 wherein the composition is utilized as a primary dressing.
60. The method of Item 57 wherein the composition is utilized as a secondary dressing.
61. The method of Item 57 wherein the composition is utilized in combination with negative pressure wound therapy.
62. The method of Item 57 wherein the composition is utilized to control, reduce or eradicate the growth of bacteria within or upon a wound.
63. The method of Item 57 wherein the composition is utilized to alter, control, reduce or eradicate the substance or function of microbial biofilm within or upon a wound.
64. The method of Item 57 wherein the composition is utilized to alter the structure and function of a bacterial biofilm within or upon a body or wound.
65. The method of Item 57 wherein the composition is utilized to alter the quorum sensing of ability of bacteria within or upon a tissue.
66. The method of Item 57 wherein the composition is utilized to alter the inflammatory response of a surgical site or wound.
67. A composition of Item 1-38 further containing an active pharmaceutical ingredient for delivery to, around, or upon normal or damaged tissues or a wound.
68. A composition according to Item 1-38 whereby a buffering agent is added to adjust the apparent pH of the system.

### EXAMPLES

### Example 1

| | | | Prior to | Final |
|---|---|---|---|---|
| | Conc | Amt (g) | Formation | Conc. (wet) |
| Lactoferrin | | 1.5 | 15% | 12.50% |
| Chitosan (HMW*) | 2.00% | 5.7 | 1.14% | 0.95% |
| Cyanamide | 10% | 2.8 | 2.80% | 2.33% |
| Glutaraldehyde | 5% | 2 | 0% | 0.83% |

All % on a w/w basis. Balance of final concentration is Water, USP.
*High molecular weight

### Closed-Cell Foam Method

1. Mix chitosan and cyanamide solutions until homogenous solution is formed.
2. Place lactoferrin in chitosan/cyanamide solution in small aliquots.
3. With the addition of each aliquot agitate vigorously to produce foamy consistency.
4. Add glutaraldehyde solution. (Alternatively, place under vacuum for 10-15 minutes.)

To produce open-cell foam, perform the above method followed by freezing of the foam material at −20° C. Lyophilize the foam until dry.

### Example 2

| | | | Prior to | Final |
|---|---|---|---|---|
| | Conc | Amt (g) | Formation | Conc. (wet) |
| Lactoferrin | | 1.5 | 15% | 12.50% |
| Chitosan (MMW*) | 2.00% | 8.5 | 1.70% | 0.92% |
| PEG 300 | 9% | 3 | 2.70% | 2.25% |
| Glutaraldehyde | 5% | 2 | 0% | 0.83% |

All % on a w/w basis. Balance of final concentration is Water, USP.
*Medium molecular weight

### Closed-Cell Foam Method

1. Mix chitosan and PEG 300 solutions until homogenous solution is formed.
2. Place lactoferrin in chitosan/PEG solution in small aliquots.
3. With the addition of each aliquot agitate vigorously to produce foamy consistency.
4. Add glutaraldehyde solution and place under vacuum for 10-15 minutes.

To produce open-cell foam, perform the above method followed by freezing of the foam material at −20° C. Lyophilize the foam until dry.

### Example 3

| | | | Prior to | Final |
|---|---|---|---|---|
| | Conc | Amt (g) | Formation | Conc. (wet) |
| Lactoferrin | | 1.5 | 15% | 12.50% |
| Chitosan (LMW*) | 2.00% | 8.5 | 1.70% | 1.41% |
| Glutaraldehyde | 5% | 2 | 0% | 0.83% |

All % on a w/w basis. Balance of final concentration is Water, USP.
*Low molecular weight

### Closed-Cell Foam Method

1. Place lactoferrin in chitosan solution in small aliquots.
2. With the addition of each aliquot agitate vigorously to produce foamy consistency.
3. Add glutaraldehyde solution and place under vacuum for 10-15 minutes.

To produce open-cell foam, perform the above method followed by freezing of the foam material at −20° C. Lyophilize the foam until dry.

### Example 4

| | | | Prior to | Final |
|---|---|---|---|---|
| | Conc | Amt (g) | Formation | Conc. (wet) |
| Lactoferrin | | 1.5 | 15% | 10.71% |
| Chitosan (MMW) | 2.00% | 6 | 1.20% | 0.86% |
| Glutaraldehyde | 2.50% | 4 | 0% | 0.71% |

All % on a w/w basis. Balance of final concentration is Water, USP.

### Closed-Cell Foam Method

1. Place lactoferrin in chitosan solution in small aliquots.
2. With the addition of each aliquot agitate vigorously to produce foamy consistency.
3. Add glutaraldehyde solution and place under vacuum for 10-15 minutes.

To produce open-cell foam, perform the above method followed by freezing of the foam material at −20° C. Lyophilize the foam until dry.

### Example 5

| | | | Prior to | Final |
|---|---|---|---|---|
| | Conc | Amt (g) | Formation | Conc. (wet) |
| Lactoferrin | | 1.5 | 15% | 9.38% |
| Chitosan (MMW) | 2.00% | 3 | 0.6% | 0.375% |
| Chitosan (LMW) | 2.00% | 3 | 0.6% | 0.375% |
| Glutaraldehyde | 1.67% | 6 | 0% | 0.63% |

All % on a w/w basis. Balance of final concentration is Water, USP.

### Closed-Cell Foam Method

1. Mix MMW chitosan solution and LMW chitosan solution with agitation.
2. Place lactoferrin in chitosan solution in small aliquots.
3. With the addition of each aliquot agitate vigorously to produce foamy consistency.
4. Add glutaraldehyde solution and place under vacuum for 10-15 minutes.

To produce open-cell foam perform, the above method followed by freezing of the foam material at −20° C. Lyophilize the foam until dry.

### Example 6

| | | | Prior to | Final |
|---|---|---|---|---|
| | Conc | Amt (g) | Formation | Conc. (wet) |
| Lactoferrin | | 1.5 | 14% | 12.50% |
| Chitosan (MMW) | 2.00% | 8.5 | 1.62% | 1.42% |
| Urea | | 0.5 | 4.76% | 4.17% |
| Glutaraldehyde | 5% | 2 | 0% | 0.83% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add glutaraldehyde solution and place under vacuum for 10-15 minutes.

To produce open-cell foam perform, the above method followed by freezing of the foam material at −20° C. Lyophilize the foam until dry.

### Example 7

| | | | Prior to | Final |
|---|---|---|---|---|
| | Conc | Amt (g) | Formation | Conc. (wet) |
| Lactoferrin | | 1.5 | 14% | 12.50% |
| Chitosan (MMW) | 2.00% | 8.5 | 1.62% | 1.42% |
| Urea | | 0.5 | 4.76% | 4.17% |
| Glutaraldehyde | 5% | 2 | 0% | 0.83% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add glutaraldehyde solution and heat to 60° C. for 2-4 hrs.

To produce open-cell foam perform, the above method followed by freezing of the foam material at −20° C. Lyophilize the foam until dry.

### Example 8

| | | | Prior to | Final |
|---|---|---|---|---|
| | Conc | Amt (g) | Formation | Conc. (wet) |
| Lactoferrin | | 1.5 | 14% | 12.50% |
| Chitosan (MMW) | 2.00% | 8.5 | 1.62% | 1.42% |
| Urea | | 0.5 | 4.76% | 4.17% |
| Glutaraldehyde | 5% | 2 | 0% | 0.83% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add glutaraldehyde solution and flash freeze followed by lyophilization.

### Example 9

| | | | |
|---|---|---|---|
| | | Prior to | Final |
| Conc | Amt (g) | Formation | Conc. (wet) |

| | | | | |
|---|---|---|---|---|
| Lactoferrin | | 1.5 | 14% | 14.29% |
| Chitosan (MMW) | 2.00% | 8.5 | 1.62% | 1.62% |
| Urea | | 0.5 | 4.76% | 4.76% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Place in mold and heat to 60° C. for 2-4 hrs.

### Example 10

| | | | Prior to | Final |
|---|---|---|---|---|
| | Conc | Amt (g) | Formation | Conc. (wet) |
| Lactoferrin | | 1.5 | 15% | 14.29% |
| Chitosan (MMW) | 2.00% | 8.5 | 1.70% | 1.62% |
| Urea | | 0.5 | 5.00% | 4.76% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Immediately freeze at −80° C.-−20° C. followed by lyophilization.

### Example 11

| | | | Prior to | Final |
|---|---|---|---|---|
| | Conc | Amt (g) | Formation | Conc. (wet) |
| Lactoferrin | | 1.5 | 14% | 9.68% |
| Chitosan (MMW) | 2.00% | 8.5 | 1.62% | 1.10% |
| Urea | | 0.5 | 4.76% | 3.23% |
| PEG 8000 | | 3 | 0% | 19.35% |
| Glutaraldehyde | 5% | 2 | 0% | 0.65% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add PEG 8000 and disperse rapidly followed immediately by glutaraldehyde solution.

To produce open-cell foam perform, the above method followed by freezing of the foam material at −20° C. Lyophilize the foam until dry.

### Example 12

| | | | Prior to | Final |
|---|---|---|---|---|
| | Conc | Amt (g) | Formation | Conc. (wet) |
| Lactoferrin | | 1.5 | 14% | 9.68% |
| Chitosan (MMW) | 2.00% | 8.5 | 1.62% | 1.10% |
| Urea | | 0.5 | 4.76% | 3.23% |
| CMC Sodium | | 3 | 0% | 19.35% |
| Glutaraldehyde | 5% | 2 | 0% | 0.65% |

All % on a w/w basis. Balance of final concentration is Water, USP.

### Closed-Cell Foam Method

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add CMC Sodium and disperse rapidly followed immediately by glutaraldehyde solution.

To produce open-cell foam perform, the above method followed by freezing of the foam material at −20° C. Lyophilize the foam until dry.

### Example 13

| | | | Prior to | Final |
|---|---|---|---|---|
| | Conc | Amt (g) | Formation | Conc. (wet) |
| Lactoferrin | | 1.5 | 15% | 12.50% |
| Chitosan (MMW) | 2.00% | 8.5 | 1.70% | 1.42% |
| Urea | | 0.5 | 5.00% | 4.17% |
| Glutaraldehyde | 5% | 2 | 0% | 0.83% |

All % on a w/w basis. Balance of final concentration is Water, USP.

### Closed-Cell Foam Method—Granules

1. Add urea to chitosan solution and mix until homogenous solution is formed.
2. Place lactoferrin in chitosan/urea solution in small aliquots.
3. With the addition of each aliquot agitate vigorously to produce foamy consistency.
4. Add mixture drop wise to 50 ml of cottonseed oil with constant stirring.
5. Allow to mix until well dispersed.
6. Add glutaraldehyde solution drop wise and continue mixing for 30-45 minutes.
7. Remove granules from oil by vacuum filtration.
8. Wash granules with 20 ml of acetone and air dry or dry lyophilize product to dryness.

### Example 14

| | | | Prior to | Final |
|---|---|---|---|---|
| | Conc | Amt (g) | Formation | Conc. (wet) |
| Lactoferrin | | 1.5 | 15% | 10.71% |
| Chitosan (MMW) | 2.00% | 5.7 | 1.14% | 0.81% |
| Na Bicarb Soln | 5% | 2.8 | 1.4% | 1% |
| Glutaraldehyde in 10% | | | | |
| Citric Acid Soln | 1.67% | 4 | 0% | 0.48% |

All % on a w/w basis. Balance of final concentration is Water, USP.

### Closed-Cell Foam Method

1. Mix chitosan and sodium bicarbonate solutions until homogenous solution is formed.
2. Place lactoferrin in chitosan/Na bicarbonate solution in small aliquots.
3. With the addition of each aliquot agitate vigorously to produce foamy consistency.
4. Add glutaraldehyde/citric acid solution and mix briefly. (Alternatively, place under vacuum for 10-15 minutes)

### Example 15

| | | | Prior to | Final |
|---|---|---|---|---|
| | Conc | Amt (g) | Formation | Conc. (wet) |
| BSA | | 1.5 | 15% | 15% |
| Chitosan (MMW) | 2.00% | 0.75 | 0.15% | 0.15% |
| Urea | | 0.5 | 5.00% | 5.00% |

All % on a w/w basis. Balance of final concentration is Water, USP.

Closed-cell foam method
1. Add urea to 7.25 g H₂0 and mix until solution is formed.
2. Place chitosan solution in urea/H₂0 and mix well.
3. Add BSA in aliquots with vigorous agitation until foam consistency is obtained.
4. Place foam in hot water bath at 70° C. for 1 hr.

To produce open-cell foam perform, the above method followed by freezing of the foam material at −20° C. Lyophilize the foam until dry.

### Example 16

| | | | Prior to | Final |
|---|---|---|---|---|
| | Conc | Amt (g) | Formation | Conc. (wet) |
| BSA | | 2.5 | 25% | 25% |
| Chitosan (MMW) | 2.00% | 0.75 | 0.15% | 0.15% |
| Urea | | 0.5 | 5.00% | 5.00% |

All % on a w/w basis. Balance of final concentration is Water, USP.

### Closed-Cell Foam Method

1. Add urea to 6.25 g H₂0 and mix until solution is formed.
2. Place chitosan solution in urea/H₂0 and mix well.
3. Add BSA in aliquots with vigorous agitation until foam consistency is obtained.
4. Place foam in hot water bath at 70° C. for 1 hr.

To produce open-cell foam perform, the above method followed by freezing of the foam material at −20° C. Lyophilize the foam until dry.

### Example 17

| | | | Prior to | Final |
|---|---|---|---|---|
| | Conc | Amt (g) | Formation | Conc. (wet) |
| BSA | | 3.5 | 35% | 35% |
| Chitosan (MMW) | 2.00% | 0.75 | 0.15% | 0.15% |
| Urea | | 0.5 | 5.00% | 5.00% |

All % on a w/w basis. Balance of final concentration is Water, USP.

### Closed-Cell Foam Method

1. Add urea to 5.25 g H₂0 and mix until solution is formed.
2. Place chitosan solution in urea/H₂0 and mix well.
3. Add BSA in aliquots with vigorous agitation until foam consistency is obtained.
4. Place foam in hot water bath at 70° C. for 1 hr.

To produce open-cell foam perform, the above method followed by freezing of the foam material at −20° C. Lyophilize the foam until dry.

### Example 18

| | | | Prior to | Final |
|---|---|---|---|---|
| | Conc | Amt (g) | Formation | Conc. (wet) |
| BSA | | 3.5 | 35% | 29.17% |
| Chitosan (MMW) | 2.00% | 0.75 | 0.15% | 0.125% |
| Urea | | 0.5 | 5.00% | 4.1% |
| Glutaraldehyde | 5% | 2 | 0% | 0.83% |

All % on a w/w basis. Balance of final concentration is Water, USP.

### Closed-Cell Foam Method

1. Add urea to 5.25 g H₂0 and mix until solution is formed.
2. Place chitosan solution in urea/H₂0 and mix well.
3. Add BSA in aliquots with vigorous agitation until foam consistency is obtained.
4. Add glutaraldehyde solution and allow to solidify. (Alternatively, place under vacuum for 10-15 minutes).

To produce open-cell foam perform, the above method followed by freezing of the foam material at −20° C. Lyophilize the foam until dry.

### Example 19

| | | | Prior to | Final |
|---|---|---|---|---|
| | Conc | Amt (g) | Formation | Conc. (wet) |
| BSA | | 1.5 | 15% | 9.375% |
| Chitosan (MMW) | 2.00% | 0.75 | 0.15% | 0.094% |
| Urea | | 0.5 | 5.00% | 3.125% |
| Glutaraldehyde | 1.67% | 6 | 0% | 0.626% |

All % on a w/w basis. Balance of final concentration is Water, USP.

### Closed-Cell Foam Method

1. Add urea to 7.25 g H₂0 and mix until solution is formed.
2. Place chitosan solution in urea/H₂0 and mix well.
3. Add BSA in aliquots with vigorous agitation until foam consistency is obtained.
4. Add glutaraldehyde solution and allow to solidify (alternatively, place under vacuum for 10-15 minutes).

To produce open-cell foam perform, the above method followed by freezing of the foam material at −20° C. Lyophilize the foam until dry.

### Example 20

| | | | Prior to | Final |
|---|---|---|---|---|
| | Conc | Amt (g) | Formation | Conc. (wet) |
| Lactoferrin | 10% | 2000 | 5% | 4.5% |
| Gelatin | 10% | 2000 | 5% | 4.5% |
| Formaldehyde | 10% | 400 | 0% | 0.91% |

All % on a w/w basis. Balance of final concentration is Water, USP.

### Porous Foam

1. Mix lactoferrin and gelatin solutions until homogenous solution is formed.
2. Begin foaming the proteinaceous blend with mechanical energy (homogenization with air is preferred to generate uniform cell structure).
3. Add aldehyde solution under continuous agitation and homogenization until complete.
4. Freeze the resulting foam in a suitable mold until lyophilizer is available.
5. Lyophilize the composition contained in the molds to dry and formalize final structure.

Note: Aldehyde may be increased to make more firm or reduced to decrease toxicity as required by scale.

### Example 21

| | | | Prior to | Final |
|---|---|---|---|---|
| | Conc | Amt (g) | Formation | Conc. (wet) |
| Lactoferrin | 10% | 4000 | 10% | 4.5% |
| Formaldehyde | 10% | 400 | 0% | 0.91% |

All % on a w/w basis. Balance of final concentration is Water, USP.

### Porous Foam

1. Mix lactoferrin and gelatin solutions until homogenous solution is formed.
2. Begin foaming the proteinaceous blend with mechanical energy (homogenization with air is preferred to generate uniform cell structure).
3. Add aldehyde solution under continuous agitation and homogenization until complete.
4. Freeze the resulting foam in a suitable mold until lyophilizer is available.
5. Lyophilize the composition contained in the molds to dry and formalize final structure.

Note: Aldehyde may be increased to make more firm or reduced to decrease toxicity as required by scale.

### Example 22

| | | | Prior to | Final |
|---|---|---|---|---|
| | Conc | Amt (g) | Formation | Conc. (wet) |
| Lactoferrin | 10% | 3200 | 8% | 4.5% |
| Gelatin | 10% | 800 | 2% | 4.5% |
| Formaldehyde | 10% | 400 | 0% | 0.91% |

All % on a w/w basis. Balance of final concentration is Water, USP.

### Porous Foam

1. Mix lactoferrin and gelatin solutions until homogenous solution is formed.
2. Begin foaming the proteinaceous blend with mechanical energy (homogenization with air is preferred to generate uniform cell structure).
3. Add aldehyde solution under continuous agitation and homogenization until complete.
4. Freeze the resulting foam in a suitable mold until lyophilizer is available.
5. Lyophilize the composition contained in the molds to dry and formalize final structure.

Note: Aldehyde may be increased to make more firm or reduced to decrease toxicity as required by scale.

We claim:

1. A solidified foam composition useful as a tissue sealant, tissue dressing, hemostatic sponge or tissue barrier comprising: lactoferrin, derivatives thereof, and any combination thereof crosslinked to solidify the structure.

2. The composition of claim 1 further comprising a secondary component selected from the group of: (a) an augmentative polymer, monomer, or compound with reactive groups, (b) an adjunct compound; (c) an anti-infective; (d) a crosslink augmentation agent; (e) a crosslinking-agent, and any combination thereof.

3. The composition of claim 2 wherein the augmentative polymer, monomer or compound contains reactive sites selected from the group of a nitrogen containing site, a sulfur containing site, or any combination thereof.

4. The composition of claim 2 wherein the adjunctive compound is selected from the group of surfactants, antioxidants, fatty acids, polyvinylpyrrolidone, polyvinyl alcohol, hydrogen peroxide, poly(ethylene glycol), carrageenen, alginates, and any combination thereof.

5. The composition of claim 2 wherein the anti-infective is selected from the group of urea, a lipid compound or compounds, fatty acids, a silver compound, lysozyme, sulfonamide, sulfamethoxazole, a sugar, a sugar alcohol, xylitol, methylene blue, gentian violet, an aminoglycoside, tetracyclines, macrolides, glycopeptides, lipoglycopeptides, beta lactams, quinolones, and any combination thereof.

6. The composition of claim 2 wherein the crosslinking-agent is selected from the group consisting of an aldehyde compound, a polyaldehyde compound, formaldehyde, glutaraldehyde, acetaldehyde, malonaldehyde, succinaldehyde, adipaldehyde, dialdehyde starch, glyoxal, glyoxylic acid, adipyldichloride, acrolein, N,N′-methylenebisacrylamide, diphenylphosphoryl azide, N,N′-ethylenebisacrylamide, diphenylphosphoryl azide, (poly)ethylene glycol di(meth)acrylate, functionalized (poly)ethylene glycol derivatives, ethylene glycol diglycidyl ether, glycidylmethacrylate, polyamidoamineepichlorohydrin, trimethylolpropanetriacrylate, piperazinediacrylamide, epichlorohydrin, 1,2-diol compounds, tannins, and any combination thereof.

7. The composition of claim 2 wherein the crosslink augmentation agent is selected from the group of polyamine compounds, polyhydroxybenzene, resorcinol, vanillin, urea, nicotinamide, adenosine, carbodiimide, cyanamide, and any combination thereof.

8. A method of producing a solidified foam useful as a tissue sealant, tissue dressing, hemostatic sponge or tissue barrier comprising: (a) dissolving lactoferrin, derivatives thereof, and any combination thereof, (b) mixing the dissolved material with at least one gas vigorously, including by homogenization, and (c) subsequently adding a crosslinking-agent to crosslink reactive sites of the composition thereby producing the desired solidified foam.

9. A method of producing a solidified multiparticulate composition useful as a biomaterial device, tissue implant and wound dressing comprising: combining (a) lactoferrin, derivatives thereof, and any combination thereof, and (b) a secondary component selected from the group of: (i) an augmentative polymer, monomer, or compound with reactive groups, (ii) an adjunct compound, (iii) an anti-infective, (iv) a crosslink augmentation agent and any combination thereof, and (c) adding a crosslinking-agent to solidify the composition for production of multiparticulates.
